(19) United States
(12) Patent Application Publication
(10) Pub. No.: US 2023/0001076 A1
(43) Pub. Date: Jan. 5, 2023

(54) METALLIC SUPPORT STRUCTURE IN THE DRIVE ARM/HEAD OF A MEDICAL FLUID PUMP

(71) Applicant: B. Braun Melsungen AG, Melsungen (DE)

(72) Inventors: Jan Schwarz, Melsungen (DE); Dirk Aljets, Wolfhagen (DE); Marcel Stich, Nieste (DE); Matthias Schwalm, Schwalmstadt (DE); Stefan Batzdorf, Fulda (DE); Joachim Schuetz, Fulda (DE)

(21) Appl. No.: 17/852,480

(22) Filed: Jun. 29, 2022

(30) Foreign Application Priority Data
Jul. 1, 2021 (DE) 20 2021 103 530.7

(51) Int. Cl. A61M 5/145; A61M 5/14

(52) U.S. Cl. CPC A61M 5/1452; A61M 5/1413; A61M 2205/502

(57) ABSTRACT

A medical fluid pump, in particular a syringe pump, includes a housing and a drive head linearly movable toward the housing via a tubular or rod-shaped drive arm. The drive head has a drive head lower shell facing the housing and a drive head upper shell turned away from the housing. A metallic support structure in the form of a plate is accommodated in an interior space formed by the drive head lower shell and the drive head upper shell and is directly fixed to the drive head lower shell and to a free end portion of the tubular or rod-shaped drive arm.

### CROSS-REFERENCE TO RELATED APPLICATION

This application claims priority under 35 U.S.C. § 119 to German Application No. 20 2021 103 530.7, filed Jul. 1, 2021, the content of which is incorporated by reference herein in its entirety.

### FIELD

The present disclosure relates to a metallic support structure that is installed in a drive head of a medical fluid pump. The drive head consists of a drive head lower shell and a drive head upper shell, and the metallic support structure is adapted to support an end/end portion of a tubular retaining/drive arm projecting into the drive head lower shell as well as a number of electronic components inside the drive head in a stable manner and without any further mechanical support elements of the drive head upper shell. In other words, the upper shell supports and, resp., bears neither the drive arm projecting into the drive lower shell nor the electronic components installed in the lower shell, i.e., the bearing of the drive arm and the electronic components installed in the lower shell takes place exclusively using the metallic support structure in connection with the drive head lower shell.

### BACKGROUND

The development in modern medicine, in particular in intensive care medicine, has resulted in infusion therapies requiring the well-targeted use and the exact dosing of highly efficient drugs via fluid pumps. A fluid pump design used very frequently in this case is the so-called syringe pump. For operating a syringe pump, a filled syringe barrel is inserted into the syringe pump and the free end of the extended syringe plunger/syringe plunger rod is fixed to a drive head attached to a retaining/drive arm. The end/end portion of the syringe plunger is usually retained on the drive head by adjustable angled arms/clips. Also, in the drive head frequently a force is measured which is exerted by a pump drive via the drive arm and the drive head mounted thereon upon the syringe plunger so that clogging can be detected in the fluid path between the syringe and the patient, for example.

Thus, the drive head of such syringe pump is a complex component part with high requirements to dimensional accuracy. Due to its arrangement effectuated by the drive arm and projecting from the pump housing, the drive head is moreover also frequently exposed, during operation, to at least minor shocks which may be result from the handling, the set-up and dismantling etc. of the pump by the operator or, in an extreme case, even by the pump falling down.

The drive head solutions known from prior art such as from WO 2012/123 417 A1 show a bearing of the drive arm in the drive head which takes place both in the lower shell and in the upper shell of the drive head. This entails the drawback that shocks acting from outside upon the drive head are transferred undiminished to the sensitive components and to the bearing of the drive arm and, thus, may result in damage. Further, the bearing via the drive head lower and upper shells entails the drawback that higher manufacturing tolerances are required and that pretensions due to the screwing of the drive head lower and upper shells may be introduced to the bearing and to the sensitive components.

### SUMMARY

The objects and purposes of the disclosure are to eliminate or at least reduce the drawbacks of prior art and to provide in particular a medical syringe pump and, resp., a metallic support structure for a drive head which allows for an exclusive bearing of the drive arm in connection with the drive head lower shell and ensures decoupling of the flux of force from the drive head lower shell to the drive head upper shell and the components installed there. Furthermore, the metallic support structure is intended to contribute to bearing components in the drive head lower shell without pretensions due to the screwed connection of the drive head lower shell and upper shell being induced in the bearing of the components.

The medical fluid pump, in particular syringe pump, comprises a housing and a drive head linearly movable to the housing via a tubular or rod-shaped drive arm or, resp., a drive arm, the drive head having a drive head lower shell facing the housing and preferably made of plastic and a drive head upper shell turned away from the housing and preferably made of plastic. Also, the syringe pump has a metallic support structure preferably in the form of a plate which is accommodated in an interior space formed by the drive head lower shell and the drive head upper shell and is directly fixed to a free end portion of the tubular or rod-shaped drive arm and preferably also to the drive head lower shell.

The direct fixation of the drive arm in the drive head lower shell offers the advantage that, in contrast to a joint bearing via the drive head lower shell and upper shell, the bearing of the drive arm remains unaffected by the fitting accuracy between the drive head lower shell and upper shell. The metallic plate shape of the support structure continues to constitute a so-called robust core in the plastic housing of the drive head and, on the one hand, by its flat shape distributes forces introduced in the drive head via the bearing of the drive arm extensively to the housing cross-section, allowing stress peaks to be avoided, and, on the other hand, the metallic plate shape serves so-to-speak as a robust partition between the drive head lower shell and upper shell. In its function as a robust partition, the metallic support structure additional shields shocks coming from the direction of the housing upper shell against the components situated in the housing lower shell.

In a preferred variant, the medical fluid pump may include a U-shaped bracket extending in the axial direction of the drive arm, the U-shaped bracket being integrally formed with the side of the drive head lower shell facing the interior space so that it encompasses the drive arm and, thus, delimits the axial insertion depth of the tubular drive arm in the drive head lower shell.

The U-shaped bracket can be formed of a circular sleeve-type portion at the inner face of the drive housing lower shell and project in the longitudinal direction of the drive arm into the drive housing. The arms of the U-shaped bracket extending in the longitudinal direction may include a through-bore for passing through a clamp sleeve on each of opposite sides. This offers the advantage that the drive arm inserted in the U-shaped bracket which also includes corresponding through-bores level with the through-bores at the arms of the U-shaped bracket can be aligned and secured radially and axially in the U-shaped bracket via a clamp sleeve in a pre-assembling step already.

In another preferred embodiment of the medical fluid pump, the metallic support structure can consist of a first portion and a second portion formed separately from the first portion, each abutting on the drive arm.

This facilitates the assembly and the alignment of the metallic support structure on the drive arm, for example.

In a particularly preferred embodiment of the medical fluid pump, the support structure can engage, via projections, in bores of the tubular drive arm and, in this way, can secure the drive head lower shell relative to the drive arm in the axial direction as well as against rotation. The projections of the support structure protruding into the drive arm advantageously help additionally provide a positive connection between the drive arm and the support structure.

In accordance with an advantageous development of the medical fluid pump, the first portion of the support structure can form, with the second portion of the support structure, a clamp for clamping the drive arm at the U-shaped bracket so that the drive arm is secured in the axial direction as well as against rotation. The clamp shape of the support structure does not only facilitate the assembly, but also enables the clamping force relative to the drive arm and the U-shaped bracket to be set via the screwing of the clamp.

In addition, the clamp formed of the first portion and the second portion of the support structure can have an inner contour reproducing the U-shaped bracket which permits axial and rotational fine alignment at the U-shaped bracket. At the same time, the reproduction of the inner contour of the U-shaped bracket can also enable, when the clamp is closed, a positive connection against the direction of rotation, i.e., along the circumference, which renders the connection between the support structure and the drive head even more robust against rotation.

Further, the first portion of the support structure can be indirectly connected to the drive head lower shell via a screwed connection. In the indirect screwing, the first portion of the support structure is connected to a flat component which in turn is supported itself in the housing via cylinder pins. In this way, pretensions between the support structure and the drive housing lower shell due to screwed connections are avoided, thereby preventing component distortion and extensively distributing bearing forces.

Furthermore, in a preferred embodiment, the first portion of the support structure may include a recess for passing through cables, thus facilitating an electronic connection between the drive arm and the components present in the drive head lower shell.

In another embodiment of the medical fluid pump, the first portion and the second portion of the support structure can be detachable from each other. This facilitates assembling and aligning the support structure in the housing lower shell.

Further, the drive head upper shell can close and fluid-tightly seal the interior space formed between the drive head lower shell and the drive head upper shell without aligning components in the drive head lower shell. The aim is to ensure that, although the drive head upper shell is connected positively and non-positively to and efficiently seals the drive housing lower shell, impacts and warping are largely absorbed via the drive housing, which is composed of the drive housing lower shell and the drive housing upper shell, and only as little as possible of the force effect is transferred to the internal components, however.

### BRIEF DESCRIPTION OF THE DRAWING FIGURES

The disclosure shall be illustrated in detail in the following by means of Figures using a preferred embodiment.

FIG. 1 is a perspective view of a syringe pump according to the invention.

FIG. 2 is a perspective view of a drive head comprising swivel arms and a drive arm.

FIG. 3 is a perspective view of the inner face of the drive head lower shell without an installed base plate.

FIG. 4 is an exploded view of the drive head lower shell as well as the components of base plate, board and board end plate to be inserted.

FIG. 5 is a perspective view of the drive head lower shell comprising the inserted components of base plate, board and board end plate as well as an exploded view of the support structure consisting of the first and second portions.

FIG. 6 is a perspective view with the inserted base plate, board and board end plate and the mounted support structure.

FIG. 7 shows an exploded view of the drive head lower shell with installed components and support structure as well as the drive head upper shell.

The Figures are schematic and serve only for the comprehension of the disclosure. Like elements are provided with like reference symbols.

### DETAILED DESCRIPTION

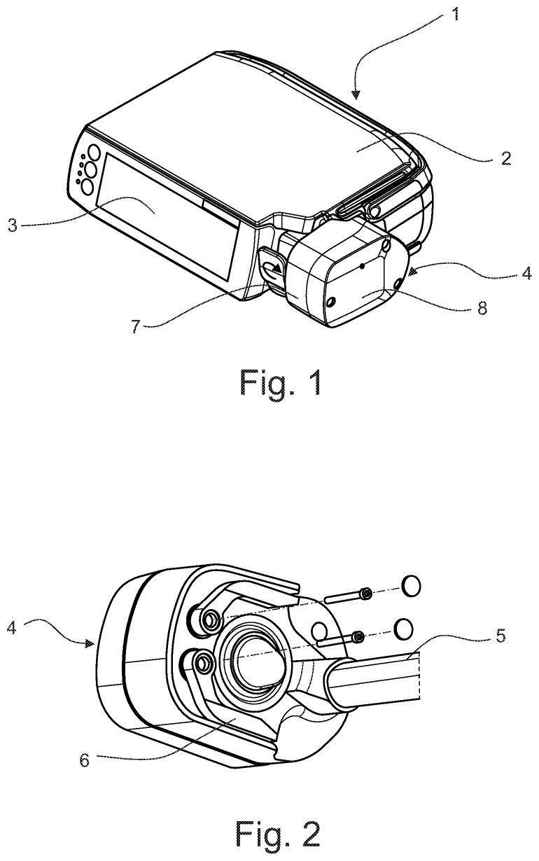

FIG. 1 illustrates a fluid pump according to the disclosure, in particular a syringe pump 1 comprising a pump housing 2 and a display 3 on the front side thereof. Laterally from the display 3 on a sidewall of the housing 2, a drive head 4 disposed substantially perpendicularly to the sidewall of the housing 2 is provided (outside the housing 2), which drive head 4 can be retracted and extended via a retaining/drive arm 5 (FIG. 2) substantially perpendicularly to the sidewall of the housing 2.

For operating the syringe pump 1, the display 3 can be unfolded and a syringe (not shown) with a filled syringe barrel can be inserted into a dedicated free space in the syringe pump 1. The free end of an extracted syringe plunger is further fixed to the drive head 4 which has been extended before via the drive arm 5 away from the sidewall of the housing 2, preferably with two swiveling angled arms 6 (FIG. 2) as a part of the drive head 4. In order to empty the syringe content in a controlled manner, the drive head 4 serving as a stop for the syringe plunger here is displaced toward the sidewall of the housing 2 preferably via a spindle drive inside the housing not shown in more detail.

As is evident from FIG. 1, the drive head 4 consists of a drive head lower shell 7 facing the housing 2 and preferably made of plastic and a drive head upper shell 8 turned away from the housing 2 and equally preferably made of plastic.

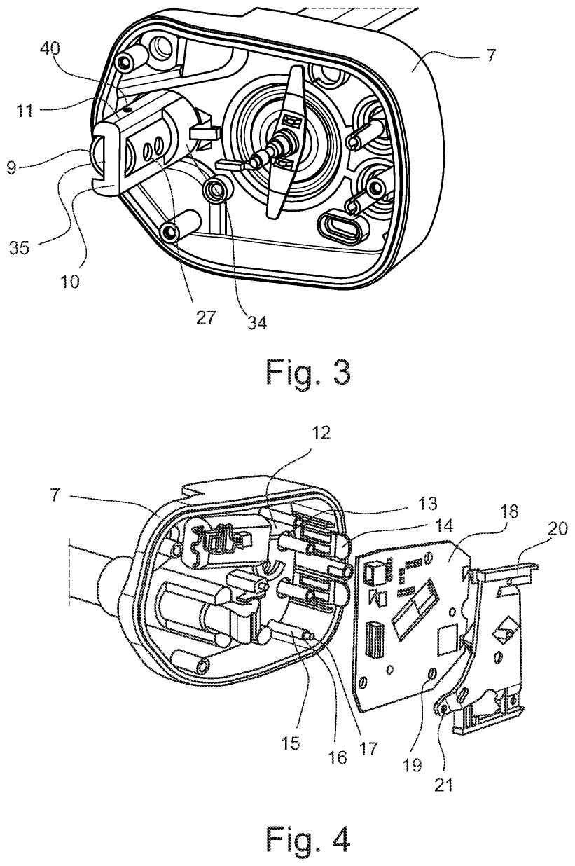

FIG. 3 illustrates the inner face of the drive head lower shell 7. As is visible, a free end portion 9 of the drive arm 5 is accommodated in a U-shaped bracket 10 serving as a stop. Further, a clamp sleeve 40 which is also passed through through-holes located at the free end portion 9 of the drive arm 5 extends transversely through the legs 11 of the U-shaped bracket 10. Using the clamp sleeve 40, the drive arm 5 is already preadjusted relative to the drive head and secured in the axial direction as well as against rotation relative to the drive head.

In FIG. 4, a base plate with electronic components 12, hereinafter merely referred to as base plate 12, inserted in the drive head lower shell 7 is shown. It is emphasized that the base plate 12 is merely inserted or attached but not screwed with the drive head lower shell 7. The base plate 12 includes three circular recesses 13 and two guide strips 14 substantially perpendicularly projecting from the base plate 12 by means of which the base plate 12 is aligned at dedicated contours of the drive head lower shell 7. Inserting the base plate 12 without screwing offers the advantage that no screwing points must be provided which might produce different stress zones on the surface of the base plate 12 and which might transfer locally concentrated shock pulses to the base plate 12 which may result in damage of the electronic components fastened on the base plate 12. At the same time, the mounting position of the base plate 12 is safeguarded by the recesses 13 and guide strips 14 which align the base plate 12 at the contours of the drive head lower shell.

Further, three substantially perpendicularly projecting cylinder pins 15 which at their free ends form a circular shoulder 16 with a cylinder pin tip 17 situated centrally therein are arranged on the base plate 12. A board 18 including three corresponding circular recesses 19 is placed, in turn, on the three shoulders 16, the recesses 19 indirectly encompassing the cylinder pin tips 17. Further, a board end plate 20 is placed onto the board 18. The board end plate 20 includes three sleeve-type extensions 21 on its side facing the board 18. The sleeve-type extensions 21 engage in the circular recesses 19 of the board 18, equally rest on the circular shoulders 16 of the cylinder pins 15 and directly enclose the cylinder pin tips 17. As the sleeve-type extensions 21 engage in the recesses 19 of the board 18 and at the same time directly encompass the cylinder pin tips 17, the board end plate 20 rests stably on the board 18 without getting out of place relative to the latter.

FIG. 5 illustrates the drive head lower shell 7 comprising the already inserted base plate 12, the board 18 and the board end plate 20. Moreover, a metallic support structure 22 is visible in an exploded view and consists of a first portion 23 and a second portion 24 both of which are provided to encompass the free end portion 9 of the drive arm 5 and the U-shaped bracket 10.

Moreover, on the end face of the first portion 23 of the support structure 22 a first cylindrical projection 25 and a second cylindrical projection 26 of a different circle diameter can be seen which are adapted to engage in two bores 27, visible in FIG. 3, and to secure the drive arm 5 relative to the drive head in the axial direction as well as against rotation in the installed state of the support structure 22 in the drive head lower shell 7.

The first portion 23 of the support structure 22 also includes, on its end face, a strip-like extension 28 with two differently sized recesses which are adapted to enable two differently sized tooth-like projections 29 at the second portion 24 of the support structure 22 to engage in the recesses of the strip-like extension 28. The side of the second portion 24 of the support structure 22 opposite to the tooth-like projections 29 in turn includes two through-bores 30 via which, using two screws 31, the second portion 24 of the support structure 22 can be screwed with the first portion 23 of the support structure 22 on the end face of which two threaded holes 32 are provided.

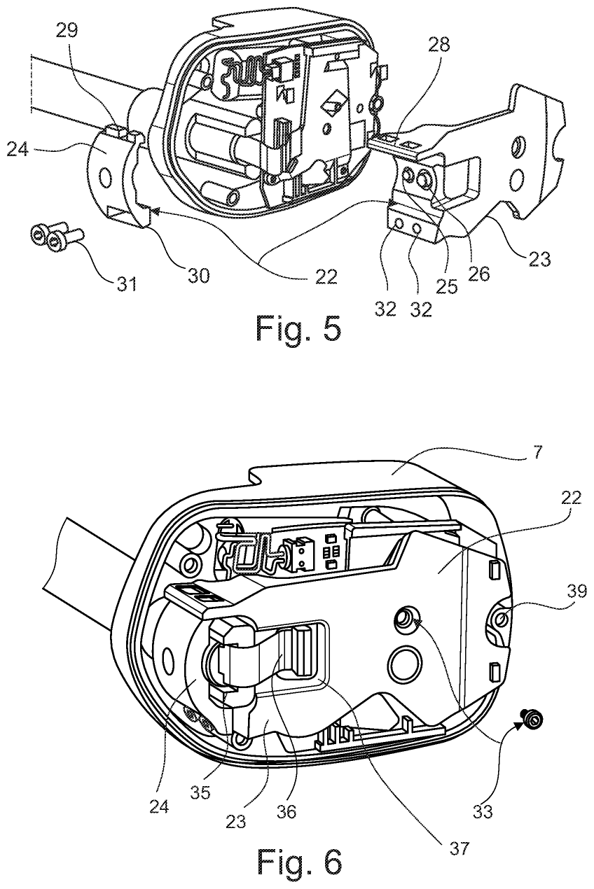

FIG. 6 shows the support structure 22 in the mounted state in the drive head lower shell 7. As is evident, the first portion 23 and the second portion 24 of the support structure 22 jointly form a clamp 22 which clamps the free end portion 9 of the drive arm 5 enclosed therein so that the drive head is secured relative to the drive arm in the axial direction as well as against rotation. It is emphasized in this context that the inner contours of the first portion 23 and the second portion 24 situated toward the free end portion 9 of the drive arm 5 reproduce the U-shaped bracket 10, thereby allowing for axial and rotational fine alignment at the U-shaped bracket 10.

As can also be seen from FIG. 6, the first portion 23 of the support structure 22 is screwed with the board end plate 20 via a screwed connection 33, resulting in the support structure 22 being indirectly connected to the drive head lower shell 7 by resting on the board end plate 20. At the same time, in the mounted state the first portion 23 and the second portion 24 of the support structure 22 directly rest, with the respective so-to-speak lower clamp side, on a shoulder of a sleeve-type portion 34 of the drive head lower shell 7, which is visible in FIG. 3.

As is evident from FIG. 6, the base plate 12, the board 18 and the board end plate 20 are held together sandwich-like via defined pressing forces from two sides, namely on the lower side by the drive head lower shell 7 and on the upper side by the first portion 23 of the support structure 22. The defined pressing forces are mainly resulting from the predetermined spaces as a result of the direct bearing faces between the support structure 22 or, resp., clamp 22 and the sleeve-type portion 34 of the drive head lower shell 7 as well as via the stacking height between the base plate 12 and the board end plate 20 which is significantly defined by the length of the cylinder pins 15.

The afore-mentioned type of assembly or the clamp-like arrangement of the drive head lower shell 7 and the support structure 22 with the interposed sensitive components which are situated above all on the base plate 12 and the board 18, offers the advantage that screwed connections can be almost completely dispensed with in shock-sensitive zones and that the pressing forces required for the cohesion of the base plate 12, the board 18 and the board end plate 20 can be distributed extensively and evenly. The support structure 22 further adopts the function of a partition from the drive head upper shell 8 and additionally protects the components situated in the drive head lower shell against impacts.

It is also visible in FIG. 6 that the U-shaped bracket 10 has on its end face a U-shaped recess 35 which serves for guiding a ribbon cable 36 which is guided through the drive arm 5. In the first portion 23 of the support structure 22, a connector recess 37 is provided which enables the ribbon cable 36 guided out of the drive arm 5 to be guided through the support structure 22 and to be connected to the board 18 underneath.

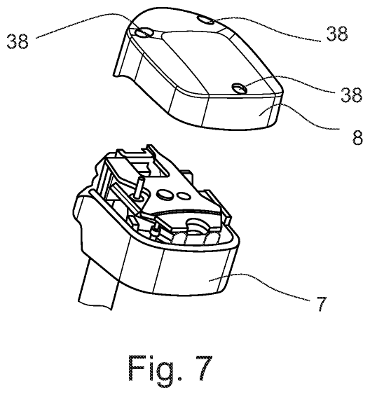

FIG. 7 illustrates an exploded view of the drive head lower shell 7 with components installed there and the support structure 22 as well as the drive head upper shell 8. The drive head upper shell 8 is screwed, via through-bores 38, with screw bosses 39 in the drive head lower shell 7 so that the drive head upper shell 8 closes and seals the drive head 4 without the drive head upper shell 8 or the screwed connections having an influence on the alignment of the base plate 12 and the board 18. Possible flows of forces caused by impacts onto the drive head upper shell 8 are thus not transferred directly to the base plate 12 and the board 18. The base plate 12 and the board 18 are quasi force-decoupled from direct force impacts of the drive head upper shell 8 and absorb them only indirectly via the bearing face in the drive head lower shell 7.

1. A medical fluid pump comprising:
a housing;
a drive arm that is tubular or rod-shaped;
a drive head linearly movable toward the housing via the drive arm; and
a metallic support structure comprising a plate,
the drive head having a drive head lower shell facing the housing and a drive head upper shell turned away from the housing,
the metallic support structure being accommodated in an interior space formed by the drive head lower shell and the drive head upper shell,
the metallic support structure being directly fixed to the drive head lower shell and to a free end portion of the drive arm.

2. The medical fluid pump according to claim 1, further comprising a U-shaped bracket extending in an axial direction of the drive arm, the U-shaped bracket being integrally formed on a side of the drive head lower shell facing the interior space so that the U-shaped bracket encompasses the drive arm and delimits an axial insertion depth of the drive arm in the drive head lower shell.

3. The medical fluid pump according to claim 1, wherein the metallic support structure comprises a first portion and a second portion formed separately from the first portion, each of the first portion and the second portion abutting on the drive arm.

4. The medical fluid pump according to claim 3, wherein the first portion and the second portion form a clamp which clamps the drive arm at the U-shaped bracket so that the drive arm is fixed in an axial direction and fixed against rotation.

5. The medical fluid pump according to claim 4, wherein the clamp has an inner contour reproducing the U-shaped bracket which permits an axial and rotational fine alignment at the U-shaped bracket.

6. The medical fluid pump according to claim 3, wherein the first portion is indirectly connected to the drive head lower shell via a screwed connection.

7. The medical fluid pump according to claim 3, wherein the first portion includes a recess for passing through cables.

8. The medical fluid pump according to claim 3, wherein the first portion and the second portion are detachable from each other.

9. The medical fluid pump according to claim 1, wherein the metallic support structure engages, via projections, in bores of the drive arm and fixes the drive head lower shell relative to the drive arm in an axial direction and fixes the drive head lower shell against rotation.

10. The medical fluid pump according to claim 1, wherein the drive head upper shell closes and fluid-tightly seals the interior space without aligning components in the drive head lower shell.

11. The medical fluid pump according to claim 1, wherein the medical fluid pump is a syringe pump.
